(19) United States
(12) Patent Application Publication
(10) Pub. No.: US 2024/0238720 A1
(43) Pub. Date: Jul. 18, 2024

(54) SYSTEMS AND METHODS TO INTEGRATE GEOTHERMAL HEAT PUMPS AND MEMBRANE SEPARATION UNITS

(71) Applicant: Saudi Arabian Oil Company, Dhahran (SA)

(72) Inventors: Yang Liu, Belmont, MA (US); Junyan Yang, Acton, MA (US)

(21) Appl. No.: 18/098,289

(22) Filed: Jan. 18, 2023

(51) Int. Cl. B01D 53/22; B01D 53/30; B01D 71/64; C07C 7/144; F25B 30/06; F25B 49/02

(52) U.S. Cl. CPC B01D 53/228; B01D 53/30; B01D 71/64; C07C 7/144; F25B 30/06; F25B 49/02; F24T 10/00

(57) ABSTRACT

The disclosure relates to systems and methods that integrate geothermal heat pumps and membrane separation units.

### FIELD

The disclosure relates to systems and methods that integrate geothermal heat pumps and membrane separation units.

### BACKGROUND

Membranes can be used for gas separations. Temperature fluctuations can affect the membrane separation performance as sorption, diffusion and membrane properties can be temperature dependent.

### SUMMARY

The disclosure relates to systems and methods that integrate thermal management using geothermal energy to control the operation temperature of a membrane separation unit. The systems and methods include controlling the temperature of a membrane separation unit using geothermal energy, which is relatively efficient, environmentally friendly, cost effective and renewable. The systems and methods can be applied in many geographical locations year-round because, in general, geothermal energy is widely available and ground temperatures are not significantly impacted by temperature fluctuations over the course of the day and/or year.

The systems and methods can reduce temperature fluctuations in the membrane separation unit, thereby reducing changes in the permeability and/or selectivity of the membrane, improving separation performance and/or improving separation efficiency, relative to certain other membrane separation methods. The systems and methods can reduce temperature fluctuations due to seasonal temperature changes, changes in temperature over the course of a day, Joule-Thompson cooling effects, and/or increases in pressure and temperature due to gas compression.

The systems and methods can have a relatively long operational lifespan compared to certain other systems and methods, such as systems and methods that include an air-source system that collect and transfer heat from the air. Improvements in separation efficiency and the relatively long lifetime of the geothermal systems are expected to provide relatively economical temperature control for membrane separation units.

The systems and methods can reduce energy use, capital costs and/or operational costs relative to certain other separation methods, such as thermally-driven separation methods (e.g., distillation) or membrane systems with a cryogenic unit. Without wishing to be bound by theory, it is believed that the electrical input of the geothermal heat pump can be offset by the enhancement of the membrane separation efficiency. Thus, the operation temperature of the membrane separation unit can be controlled relatively efficiency and economically with relatively low carbon emissions.

The systems and methods can be applied to a wide range of gas separations, such as hydrocarbon separations and sour gas treatment.

In a first aspect, the disclosure provides a system, including a membrane separation unit, and a geothermal heat pump. The geothermal heat pump includes a first heat conducting fluid loop in thermal contact with the membrane separation unit. The geothermal heat pump is configured to control a temperature of the membrane separation unit.

In some embodiments, the first heat conducting fluid loop is in direct physical contact with the membrane separation unit.

In some embodiments, the system further includes a heat distribution unit. The membrane separation unit is disposed in the heat distribution unit. The membrane separation unit is in thermal contact with the heat distribution unit. The first heat conducting fluid loop is in thermal contact with the heat distribution unit.

In some embodiments, the heat distribution unit includes copper beads.

In some embodiments, the geothermal heat pump is configured to heat the membrane separation unit.

In some embodiments, the geothermal heat pump is configured to cool the membrane separation unit.

In some embodiments, the geothermal heat pump is configured to maintain a temperature of the membrane separation unit within 10% of a desired temperature.

In some embodiments, the system further includes an earth connection system configured to exchange heat with a subterranean zone and the geothermal heat pump.

In some embodiments, the earth connection system includes a second heat conducting fluid loop in direct thermal communication with the subterranean zone and a component of the geothermal heat pump.

In some embodiments, the second heat conducting fluid loop includes a vertical closed loop heat exchange system, a horizontal closed loop heat exchange system, and/or an open loop heat exchange system.

In some embodiments, the membrane separation unit includes an interior having first and second regions, a membrane separating the first and second regions, a first inlet in fluid communication with the first region, a first outlet in fluid communication with the first region, and a second outlet in fluid communication with the second region. The membrane is configured so that, during use a gas mixture including first and second gases enters the first region via the first inlet, the first gas does not permeate the membrane and exits the membrane separation unit via the first outlet, and the second gas permeates the membrane to enter the second region and exits the membrane separation unit via the second outlet.

In some embodiments, the system further includes a controller. The controller controls the geothermal heat pump to control a temperature of the membrane separation unit.

In some embodiments, the controller further includes a temperature probe configured to measure the temperature of the membrane separation unit.

In a second aspect, the disclosure provides a method, including controlling a temperature of a membrane separation unit using a geothermal heat pump. The geothermal heat pump includes a heat conducting fluid loop in thermal contact with the membrane separation unit.

In certain embodiments, controlling the temperature of the membrane separation unit using the geothermal heat pump includes maintaining the temperature of the membrane separation unit within 10% of a desired temperature of the membrane separation unit.

In certain embodiments, the method further includes measuring a temperature of the membrane separation unit, and determining a variance from the desired temperature of the membrane separation unit.

In certain embodiments, the method further includes adjusting the temperature of the membrane separation unit using the geothermal heat pump based on the variance from the desired temperature of the membrane separation unit.

In certain embodiments, the method further includes contacting a first gas mixture including a first gas with a first side of a membrane of the membrane separation unit. At least a portion of the first gas permeates the membrane.

In certain embodiments, the first gas includes carbon dioxide, nitrogen, and oxygen, and the first gas mixture includes a hydrocarbon.

In certain embodiments, the first gas includes CO₂, the first gas mixture includes CH₄, and the membrane includes a CO/CH₄ selectivity of at least 10.

### BRIEF DESCRIPTION OF THE FIGURES

FIG. 1 depicts a schematic of a system.

FIG. 2 depicts a schematic of a system.

FIG. 3 depicts a schematic of a system.

FIG. 4 depicts a schematic of a system.

FIG. 5A depicts a schematic of a vertical closed loop heat exchange system.

FIG. 5B depicts a schematic of a horizontal closed loop heat exchange system.

FIG. 5C depicts a schematic of an open loop heat exchange system.

FIG. 6 depicts a graph of CO₂/CH₄ selectivity as a function of CO₂ permeability at several temperatures.

### DETAILED DESCRIPTION

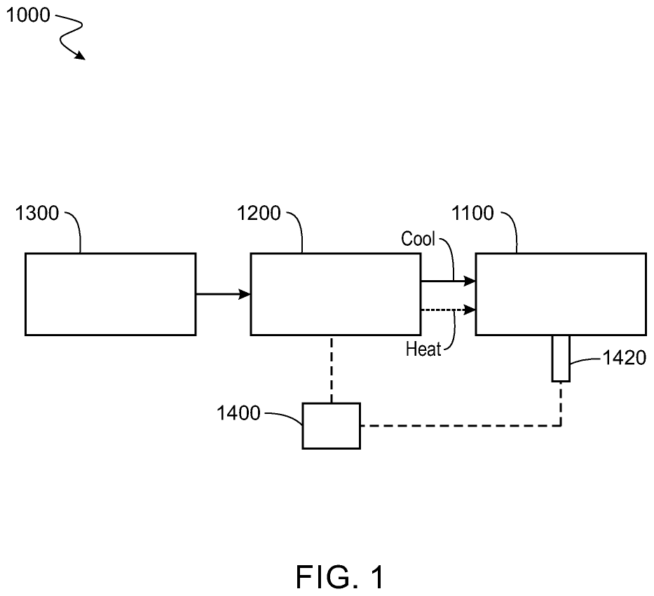

FIG. 1 depicts a system 1000 that includes a membrane separation unit 1100, a geothermal heat pump 1200 and an earth connection system 1300. The earth connection system 1300 facilitates heat extraction from the ground via a heat exchanger loop for carrying energy to the geothermal heat pump 1200 (see discussion below). The geothermal heat pump 1200 can then exchange heat with the membrane separation unit 1100, thereby controlling the temperature of the membrane separation unit (see discussion below). The geothermal heat pump 1200 can either heat or cool the membrane separation unit 1100 as desired. The system 1000 controls the temperature of the membrane separation unit 1100 to provide relatively constant separation performance during operation.

The system 1000 further includes a controller 1400, capable of controlling the geothermal heat pump 1200. The controller can include a temperature probe 1420 to monitor a temperature of the membrane separation unit 1100 and control the geothermal heat pump 1200 accordingly to maintain a desired temperature of the membrane separation unit 1100.

In certain embodiments, the desired temperature of the membrane separation unit 1100 is at least 10 (e.g., at least 20, at least 30, at least 40) ° C. and/or at most 50 (e.g., at most 40, at most 30, at most 20) ° C. In certain embodiments, the fluctuation from the desired temperature is at most 3 (e.g., at most 2, at most 1) ° C. Without wishing to be bound by theory, it is believed the fluctuation from the desired temperature is dependent on the efficiency of the geothermal heat pump 1200.

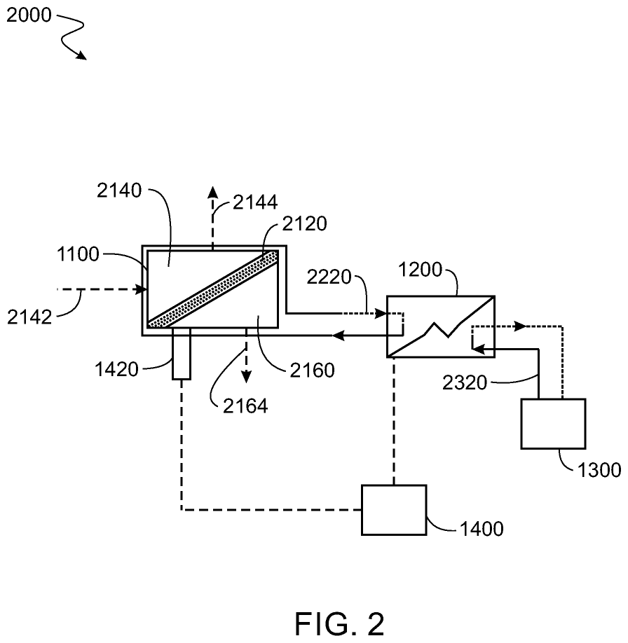

FIG. 2 depicts a system 2000 that includes the membrane separation unit 1100, the geothermal heat pump 1200 and the earth connection system 1300. The membrane separation unit 1100, includes a membrane 2120 that separates a retentate side 2140 from a permeate side 2160. The retentate side 2140 includes an inlet 2142 and an outlet 2144. The permeate side 2160 includes an outlet 2164.

The geothermal heat pump 1200 includes a heat conducting fluid loop 2220 in thermal communication with the membrane separation unit 1100. The heat conducting fluid loop 2220 physically contacts the membrane separation unit 1100. In some embodiments, the heat conducting fluid loop 2220 includes micropipes that surround the membrane separation unit 1100 to improve the thermal conduction efficiency.

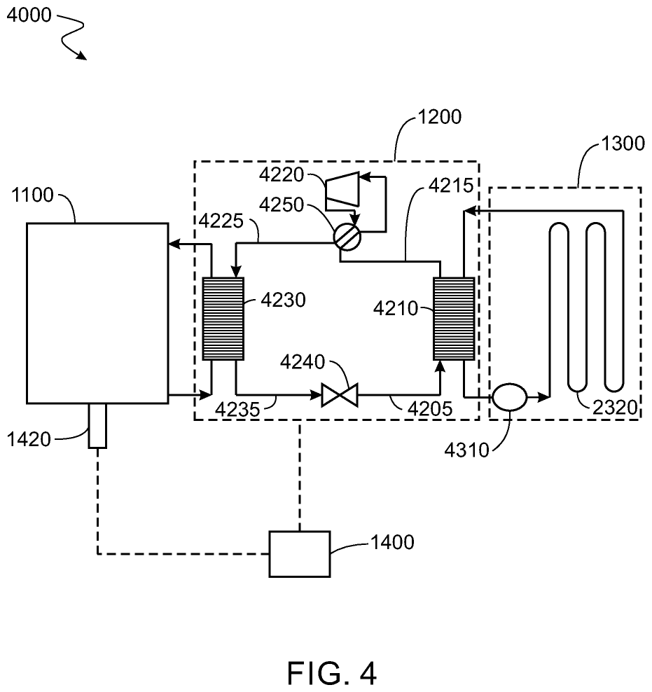

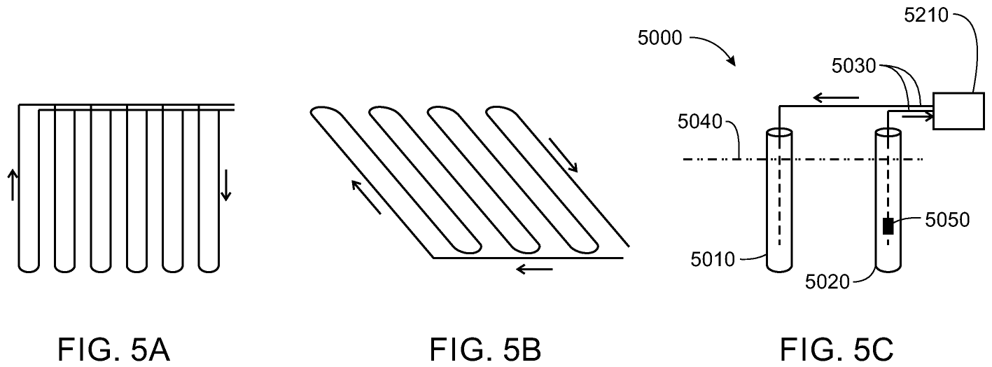

The earth connection system 1300 includes a heat conducting fluid loop 2320 to exchange heat with the geothermal heat pump (see discussion regarding FIGS. 4 and 5A-C below).

To perform a separation, an initial gas mixture containing one or more gases is introduced into the retentate side 2140 of the membrane separation unit 1100 via the inlet 2142. One or more gases in the initial gas mixture permeate the membrane 2120 to enter the permeate side 2160 to provide a permeate gas or permeate gas mixture. Any gases in the initial gas mixture that do not permeate the membrane 2120 form a retentate gas or gas mixture on the retentate side 2140 that can be removed from the membrane separation unit 1100 via the outlet 2144. The permeate gas or gas mixture can be removed from the permeate side 2160 via the outlet 2164, and the retentate gas or gas mixture can be removed from the retentate side 2140 via the outlet 2144. During this process, the geothermal heat pump 1200 reduces (e.g., prevents) temperature fluctuations of the membrane separation unit 1100 by controlling the temperature of the membrane separation unit 1100 by either heating the unit 100 or cooling the unit 1100 as appropriate.

The system 2000 further includes the controller 1400, capable of controlling the geothermal heat pump 1200. The controller includes the temperature probe 1420 to monitor the temperature of the membrane separation unit 1100 and to control the geothermal heat pump 1200 to maintain a desired temperature of the membrane separation unit 1100. In some embodiments, if the temperature of the membrane separation unit 1100 fluctuates from a desired temperature (e.g., by at least 10%, at least 15%, at least 20%, at least 25%), the controller 1400 adjusts the temperature of the membrane separation unit 1100 using the geothermal heat pump 1200 to either add heat to the membrane separation unit 1100 or to remove heat from the membrane separation unit 1100.

In some embodiments, the geothermal heat pump 1200 can heat the membrane separation unit 1100 to offset a temperature change induced by a Joule-Thompson effect (e.g., by passing the permeate gas or gas mixture through the membrane 2120). In some embodiments, the geothermal heat pump 1200 can remove heat from the membrane separation unit 1100 (e.g., heat introduced to the membrane separation unit by the atmosphere, compression and/or gas sorption).

Examples of gases that can be present in the permeate gas or gas mixture include carbon dioxide, nitrogen, and oxygen. In certain embodiments, the initial gas mixture includes a produced gas, natural gas, and/or a hydrocarbon such as methane. In certain embodiments, the retentate gas or gas mixture includes a produced gas, natural gas, a hydrocarbon such as methane, nitrogen and/or oxygen.

Examples of suitable membranes 2120 include a polymer membrane, a zeolite membrane, a metal organic framework membrane, and a graphene oxide membrane. In some embodiments, the membrane 2120 is a polymer membrane. Without wishing to be bound by theory, it is believed that the separation efficiency of polymer membranes correlates to the movement of penetrant molecules (diffusion) that is dependent on thermally activated chain motion and solubility of the penetrant molecules (sorption) which are tied to polymer-penetrant interactions and condensability. Therefore, without wishing to be bound by theory, it is believed that temperature dependent properties of the polymer material such as chain stiffness, free volume, and polymer-penetrant interactions can have a relatively strong impact on the separation performance. Thus, it is believed that temperature control can improve membrane performance of polymer membranes.

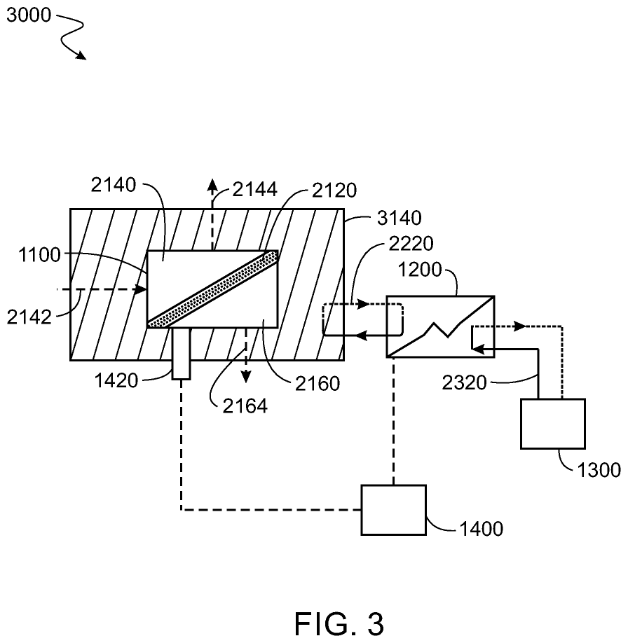

FIG. 3 depicts a system 3000 that includes the components of the system 2000. However, unlike the system 2000, the heat conducting fluid loop 2220 does not physically contact the membrane separation unit 1100 to provide direct thermal communication between the membrane separation unit 1100 and the geothermal heat pump 1200. Instead, the system 3000 includes a heat distribution unit 3140 that surrounds the membrane separation unit 1100 and that thermally contacts both the membrane separation unit 1100 and the heat conducting fluid loop 2220, allowing for relatively uniform heat distribution across the membrane separation unit 1100. In addition to the temperature of the membrane separation unit 1100, the temperature of the heat distribution unit 3140 is controlled (as described above).

In general, materials for the heat distribution unit 3140 can be selected as appropriate. Examples of the heat distribution unit 3140 include materials having a relatively high thermal conductivity, such as copper beads.

Without wishing to be bound by theory, it is believed that the system 1000, 2000 and/or 3000 can demonstrate enhanced selectivity for CO₂/CH₄ due to the relatively stable operating temperature of the membrane separation unit 1100 relative to certain other systems that do not include temperature control. In certain embodiments, the selectivity for CO₂/CH₄ is at least 10 (e.g., at least 20, at least 30, at least 40, at least 50, at least 60, at least 70, at least 80, at least 90) and/or at most 100 (e.g., at most 90, at most 80, at most 70, at most 60, at most 50, at most 40, at most 30, at most 20). Additionally or alternatively, without wishing to be bound by theory, it is believed that the system 1000, 2000 and/or 3000 can demonstrate enhanced selectivity for H₂S/CH₄ due to the relatively stable operating temperature of the membrane separation unit 1100 relative to certain other systems that do not include temperature control. In certain embodiments, the selectivity of H₂S/CH₄ is at least 20 (e.g., at least 30, at least 40, at least 50, at least 60, at least 70, at least 80, at least 90, at least 100, at least 110, at least 120, at least 130, at least 140, at least 150, at least 160, at least 170, at least 180, at least 190) and/or at most 200 (e.g., at most 190, at most 180, at most 170, at most 160, at most 150, at most 140, at most 130, at most 120, at most 110, at most 100, at most 90, at most 80, at most 70, at most 60, at most 50, at most 40, at most 30).

FIG. 4 depicts a system 4000 that includes the membrane separation unit 1100, the geothermal heat pump 1200 and the earth connection system 1300. The earth connection system includes the heat conducting fluid loop 2320 that includes a fluid. The fluid is circulated to exchange heat with the ground and the geothermal heat pump 1200. A circulation pump 4310 that reinjects the fluid in the heat conducting fluid loop 2320 back into the ground.

When used for heating the membrane separation unit 1100, thermal energy is extracted from the earth through the heat conducting fluid loop 2320 and transported to a heat exchanger 4210 of the geothermal heat pump 1200. Cold refrigerant in a liquid dominated liquid/vapor state enters the heat exchanger 4210 via the line 4205 where heat from the earth connection system 1300 is transferred to the cold refrigerant, causing the refrigerant to boil to a low-pressure vapor. The low-pressure refrigerant vapor exits the heat exchanger 4210 via the line 4215, bypasses a valve 4250 (described below), and enters a compressor 4220 (e.g., an electrically-driven compressor). The compressor 4220 increases the vapor pressure of the low-pressure refrigerant vapor. The increased-pressure refrigerant vapor then exits the compressor 4220, bypasses the valve 4250, and enters a heat exchanger 4230 via the line 4225. The heat exchanger 4230 induces heat transfer from the increased-pressure refrigerant vapor to the membrane separation unit 1100. In the heat exchanger 4230, the refrigerant vapor cools and condenses, which yields a fluid (e.g., liquid) refrigerant. The fluid (e.g., liquid) refrigerant exits the heat exchanger 4230 via the line 4235 and passes through an expansion valve 4240, which reduces the pressure and temperature of the fluid (e.g., liquid) refrigerant. The fluid (e.g., liquid) refrigerant exits the expansion valve 4240 via the line 4205 and enters the heat exchanger 4210 to repeat the cycle. After exchanging heat with the cold refrigerant from the line 4205 in the heat exchanger 4210, the fluid (e.g., liquid) in the heat conducting fluid loop 2320 is recirculated by the circulation pump 4310 to obtain additional heat from the earth.

When cooling the membrane separation unit 1100, the geothermal heat pump 1200 and earth connection system 1300 are used to transfer thermal energy from the membrane separation unit 1100 to the ground. This can be achieved by reversing the valve 4250, which reverses the flow of refrigerant fluid (e.g., liquid) and vapor in the geothermal heat pump 1200. Fluid (e.g., liquid) refrigerant enters the heat exchanger 4230 via the line 4235. Heat from the membrane separation unit 1100 is transferred to the refrigerant in the heat exchanger 4230, causing the fluid (e.g., liquid) refrigerant to boil to a vapor. The refrigerant vapor exits the heat exchanger 4230 via the line 4225, bypasses the valve 4250 and enters the compressor 4220. The compressor 4220 increases the vapor pressure of the refrigerant vapor from the line 4225. The refrigerant vapor then exits the compressor 4220, bypasses the valve 4250, and enters a heat exchanger 4210 via the line 4215. The heat exchanger 4210 induces heat transfer from the refrigerant vapor to the heat conducting fluid loop 2320 of the earth connection system 1300. In the heat exchanger 4210, the refrigerant vapor cools and condenses to yield a fluid (e.g., liquid) refrigerant. The fluid (e.g., liquid) refrigerant exits the heat exchanger 4210 via the line 4205 and passes through the expansion valve 4240, which reduces the pressure and temperature of the fluid (e.g., liquid) refrigerant. The fluid (e.g., liquid) refrigerant exits the expansion valve 4240 via the line 4235 and enters the heat exchanger 4230 to repeat the cycle. After exchanging heat with the refrigerant vapor from the line 4215 in the heat exchanger 4210, the fluid (e.g., liquid) in the heat conducting fluid loop 2320 is recirculated by the circulation pump 4310 to relinquish heat to the earth.

The system 4000 further includes the controller 1400, capable of controlling the geothermal heat pump 1200. The controller includes the temperature probe 1420 to monitor the temperature of the membrane separation unit 1100 and to control the geothermal heat pump 1200 to maintain a desired temperature of the membrane separation unit 1100. In some embodiments, if the temperature of the membrane separation unit 1100 fluctuates from a desired temperature (e.g., by at least 10%, at least 15%, at least 20%, at least 25%), the controller 1400 adjusts the temperature of the membrane separation unit 1100 using the geothermal heat pump 1200 to either add heat to the membrane separation unit 1100 or to remove heat from the membrane separation unit 1100.

The geothermal heat pump 1200 can include one or more compressors 4220. Typically, the geothermal heat pump 1200 uses electricity to drive the one or more compressors 4220 to provide work to concentrate and transport thermal energy. Generally, the geothermal heat pump 1200 is relatively efficient in its use of energy as it uses heat energy from the ground, e.g., the geothermal heat pump can have a relatively high coefficient of performance as defined by dividing the amount of the energy output by the amount of electricity input. In some embodiments, the coefficient of performance of the geothermal heat pump 1200 is at least 3 (e.g., at least 4, at least 5) and/or at most 6 (e.g., at most 5, at most 4), indicating that e.g., six units of heat output power (the amount of energy output) can be generated by every unit of electrical input power. Thus, the electrical input into the geothermal heat pump 1200 can be offset by the relatively large improvement of the membrane separation efficiency of the membrane separation unit 1100.

Typically, the heat conducting fluid loop 2320 of the earth connection system 1300 includes pipes that transfer fluid between the geothermal heat pump 1200 and the earth. In some embodiments, the ground loop can include double loop and single loop configurations. Depending, for example, on the energy source, such as soil or ground water, closed or open loop systems can be used, respectively.

FIG. 5A depicts a schematic of a vertical closed loop heat exchange system that can be used as the heat conducting fluid loop 2320. FIG. 5B depicts a schematic of a horizontal closed loop heat exchange system that can be used as the heat conducting fluid loop 2320.

FIG. 5C depicts an open loop heat exchange system 5000 with an injection well 5010 and a production well 5020 that can be used as the heat conducting fluid loop 2320. The pipes 5030 extend below the water table 5040 to exchange heat with subterranean water. A pump 5050 pumps water from the production well 5020 to a heat exchanger 5210 via the pipes 5030. The heat of the water is exchanged in the heat exchanger 5210, and the water is then sent back to the injection well 5010 via the pipe 5030.

### Example—Effect of Temperature on Membrane Separation of CO₂/CH₄

Membrane permeation was performed using a pressure-decay method using 6FDA ((4,4′-hexafluoroisopropylidene) diphthalic anhydride)-DAM (diaminomesitylene) polyimide membrane. 6FDA-DAM polyimide (Akron Polymer System) was dried in a vacuum oven at 100° C. for at least 12 hours before being dissolved in tetrahydrofuran (THF) to form a 15 wt. % polyimide/THF mixture. The solution was mixed on a rolling mixer overnight to dissolve the polyimide. The resulting casting solution was poured onto a glass plate, which was placed in a glove bag pre-saturated with THE vapor for 4 hours. 6FDA-DAM dense film (˜60-80 μm) was formed by simple casting using a draw knife with appropriate specific clearance. The films were left in a glove bag for 12 hours followed by annealing in an oven at 200° C. for 24 hrs. The thickness of the films were ˜80 μm.

Gas permeation measurements were conducted in a variable pressure, constant-volume apparatus. The membrane was housed between an upstream, capable of high-pressure gas introduction, and a downstream, which was kept under vacuum until experiments were initiated. CO₂ and CH₄ single gases were used in the measurements with feed pressures of 20 psig and 60 psig, respectively. The separations were performed at membrane separation unit temperatures of 338 K (T1), 298 K (T2), 273 K (T3), and 233 K (T4). The separations were performed for at least 4 hours to ensure the data was obtained under steady state.

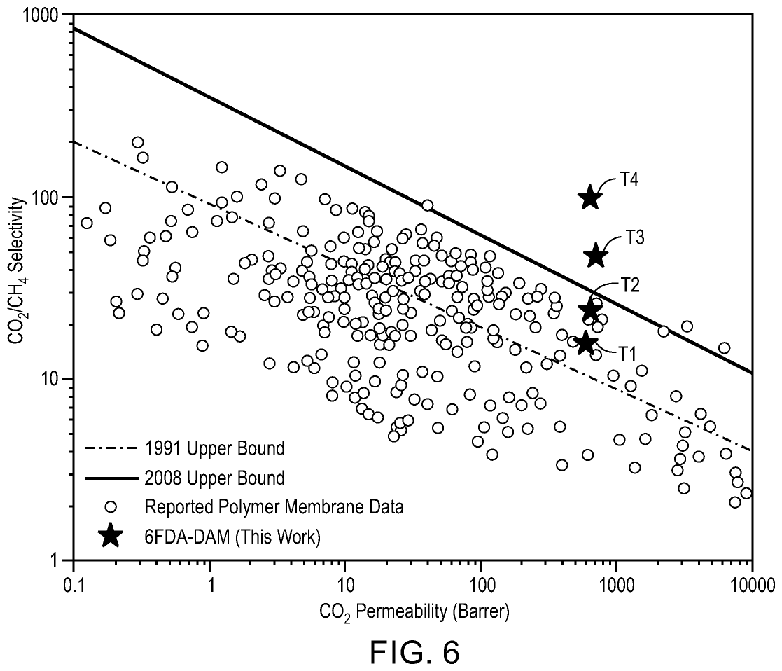

FIG. 6 depicts a graph of CO₂/CH₄ selectivity as a function of CO₂ permeability at several temperatures. As shown in FIG. 6, increasing the operation temperature from 298 K (T2, room temperature) to 338 K (T1) results in a CO₂/CH₄ selectivity reduction of ˜41.7%, whereas decreasing the temperature to 233 K (T4) increased the CO₂/CH₄ selectivity by 2.7 times. The upper bounds (dashed and solid lines) represent empirical trade-off limits of polymeric membranes for CO₂/CH₄ separation derived by Robson. Table 1 shows the CO₂ permeability, CH₄ permeability and CO₂/CH₄ selectivity measured at 338 K (T1), 298 K (T2), 273 K (T3), and 233 K (T4). The results demonstrate the importance of controlling the membrane operation temperature to provide stable separation performance.

TABLE 1
CO₂/CH₄ Separation Performance of 6FDA-DAM
Membranes at Different Temperatures.

| | CO₂ | CH₄ | CO₂/CH₄ |
|---|---|---|---|
| Temperature | Permeability | Permeability | Selectivity |
| 338 ± 2 K (T1) | 597 Barrer | 39 Barrer | 15.5 |
| 298 ± 2 K (T2) | 660 Barrer | 27 Barrer | 24.2 |
| 273 ± 2 K (T3) | 705 Barrer | 15 Barrer | 47.4 |
| 233 ± 2 K (T4) | 632 Barrer | 6.4 Barrer | 98.3 |

### Other Embodiments

While certain embodiments have been disclosed above, the disclosure is not limited to such embodiments.

As an example, in some embodiments, the systems and methods can be applied in offshore membrane separations, where the relatively constant temperature of seawater is used to control the temperature of the membrane separation unit 1100.

As another example, while embodiments have been disclosed that include the components of the systems 1000, 2000, 3000, and 4000; the disclosure is not limited to such embodiments. For example, the system 1000, 2000, 3000, and/or 4000 can contain one or more additional components not depicted. Additionally, or alternatively, the system 1000, 2000, 3000, and/or 4000 may not contain each component depicted. Further, components of the system 1000, 2000, 3000, and/or 4000 may be reconfigured as appropriate. In some embodiments, the system 1000, 2000, 3000, and/or 4000 can include more than one (e.g., at least two, at least three, at least four) temperature probes to monitor the temperature of the membrane separation unit 1100 and control the geothermal heat pump 1200 accordingly to maintain a desired temperature of the membrane separation unit 1100.

What is claimed:

1. A system, comprising:
a membrane separation unit; and
a geothermal heat pump comprising a first heat conducting fluid loop in thermal contact with the membrane separation unit,
wherein the geothermal heat pump is configured to control a temperature of the membrane separation unit.

2. The system of claim 1, wherein the first heat conducting fluid loop is in direct physical contact with the membrane separation unit.

3. The system of claim 1, further comprising a heat distribution unit, wherein:
the membrane separation unit is disposed in the heat distribution unit;
the membrane separation unit is in thermal contact with the heat distribution unit; and
the first heat conducting fluid loop is in thermal contact with the heat distribution unit.

4. The system of claim 3, wherein the heat distribution unit comprises copper beads.

5. The system of claim 1, wherein the geothermal heat pump is configured to heat the membrane separation unit.

6. The system of claim 1, wherein the geothermal heat pump is configured to cool the membrane separation unit.

7. The system of claim 1, wherein the geothermal heat pump is configured to maintain a temperature of the membrane separation unit within 10% of a desired temperature.

8. The system of claim 1, further comprising an earth connection system configured to exchange heat with a subterranean zone and the geothermal heat pump.

9. The system of claim 8, wherein the earth connection system comprises a second heat conducting fluid loop in direct thermal communication with the subterranean zone and a component of the geothermal heat pump.

10. The system of claim 9, wherein the second heat conducting fluid loop comprises a member selected from the group consisting of a vertical closed loop heat exchange system, a horizontal closed loop heat exchange system, and an open loop heat exchange system.

11. The system of claim 1, wherein the membrane separation unit comprises:
an interior having first and second regions;
a membrane separating the first and second regions;
a first inlet in fluid communication with the first region;
a first outlet in fluid communication with the first region;
a second outlet in fluid communication with the second region; and
the membrane is configured so that, during use:
a gas mixture comprising first and second gases enters the first region via the first inlet;
the first gas does not permeate the membrane and exits the membrane separation unit via the first outlet; and
the second gas permeates the membrane to enter the second region and exits the membrane separation unit via the second outlet.

12. The system of claim 1, further comprising a controller, wherein the controller controls the geothermal heat pump to control a temperature of the membrane separation unit.

13. The system of claim 12, wherein the controller further comprises a temperature probe configured to measure the temperature of the membrane separation unit.

14. A method, comprising:
controlling a temperature of a membrane separation unit using a geothermal heat pump,
wherein the geothermal heat pump comprises a heat conducting fluid loop in thermal contact with the membrane separation unit.

15. The method of claim 14, wherein controlling the temperature of the membrane separation unit using the geothermal heat pump comprises maintaining the temperature of the membrane separation unit within 10% of a desired temperature of the membrane separation unit.

16. The method of claim 15, further comprising:
measuring a temperature of the membrane separation unit; and
determining a variance from the desired temperature of the membrane separation unit.

17. The method of claim 16, further comprising adjusting the temperature of the membrane separation unit using the geothermal heat pump based on the variance from the desired temperature of the membrane separation unit.

18. The method of claim 14, further comprising:
contacting a first gas mixture comprising a first gas with a first side of a membrane of the membrane separation unit,
wherein at least a portion of the first gas permeates the membrane.

19. The method of claim 18, wherein:
the first gas comprises a first member selected from the group consisting of carbon dioxide, nitrogen, and oxygen; and
the first gas mixture comprises a hydrocarbon.

20. The method of claim 18, wherein:
the first gas comprises CO₂;
the first gas mixture comprises CH₄; and
the membrane comprises a CO/CH₄ selectivity of at least 10.
